(19) United States
(12) Patent Application Publication
(10) Pub. No.: US 2015/0351893 A1
(43) Pub. Date: Dec. 10, 2015

(54) Tissue Graft Storage Solutions And Systems

(71) Applicant: DCI DONOR SERVICES, INC., NASHVILLE, TN (US)

(72) Inventors: David Ayres Bowden Smith, Golden, CO (US); David William Duncan, Carlsbad, CA (US); Peter Alan Jenkins, Brentwood, TN (US); Harry Keith Johnson, Nashville, TN (US); Rajyalakshmi Manda, Nashville, TN (US)

(21) Appl. No.: 14/762,169

(22) PCT Filed: May 11, 2015
(86) PCT No.: PCT/US2015/030216

§ 371 Date: Jul. 20, 2015

Related U.S. Application Data
(60) Provisional application No. 61/991,107, filed on May 9, 2014.

(51) Int. Cl. A61F 2/00; A61F 2/28; B65B 5/04; B65D 77/04; B65D 81/22

(52) U.S. Cl. CPC A61F 2/0095; B65D 77/0446; B65D 81/22; B65B 5/04; A61F 2/28

(57) ABSTRACT

A system for storing an implantable device prior to a surgical implant procedure, comprising a sealable container and housed therein: a volume of solution, the solution comprising water/saline, and calcium chloride or sodium bicarbonate; and said implantable device suspended in the solution.

### TECHNICAL FIELD

The presently-disclosed subject matter relates to solutions for storing an implantable device such as tissue. More specifically, the presently-disclosed subject matter relates to aqueous solutions for storing an implantable device such as tissue for transplant in a hydrated state as well as systems for containing the tissue stored in solution.

### BACKGROUND OF THE INVENTION

The traditional method of preserving musculoskeletal grafts, such as allografts, is by the process of freeze-drying (i.e., lyophilizing) the graft. This process involves slow freezing the processed tissue while slowly drawing a vacuum on a chamber in which the tissue grafts have been placed. This process removes the water content by sublimation without forming large ice crystals that may damage the tissue. By driving the residual moisture in the grafts to 6% or lower, microbial growth (e.g., bacterial and fungal) can be halted and enzymatic degradation can be slowed by several orders of magnitude.

However feeze-dried (i.e., lyophilized) grafts require hydration before implantation. This can take up to four hours for large grafts, such as massive proximal femoral allografts used for hip reconstruction after resection of a bony tumor. For smaller grafts, such as those used for spinal surgery, 10 to 60 minutes or more can be required to properly hydrate the graft before implantation into the subject. For this reason, lyophilized grafts are often not hydrated properly in the surgical theater prior to implantation due to time constraints and other factors. Lyophilized grafts can also have diminished strength (i.e., the force required to break the bone) and diminished toughness (less resistance to fracture, i.e., more brittle), making them prone to cracking if not adequately hydrated.

Furthermore, once cracks form in a bone graft, they typically continue to grow unless solid bony fusion occurs first. Sometimes such cracking leads to no complications in the subject, but a cracked graft can also lead to unstable constructs in the spine or other bony region. A cracked graft may necessitate revision surgery. In some instances a cracked graft can collapse and/or shift in the surgical site, and may result in neurological and/or vascular injury to the subject.

This scenario can present various problems. For example, grafts can crack while the surgeon is inserting them into the surgical site, which sometimes requires tapping with a mallet. Grafts can also crack after implantation. Accordingly, it is desirable to ensure that grafts are fully hydrated prior to being implanted to maximize the material toughness and to minimize the potential for cracking.

Additionally, precision cut and CNC-machined bone grafts can shrink upon lyophillization and then expand upon hydration. The shrink factors are anisotropic; that is, they are a function of direction of the axis of the bone from which they were cut. For example, with long bones (e.g., cortical bone) the shrink factors in the circumferential and radial directions of the long bone will typically be similar and the shrink factor in the longitudinal direction can be substantially different. For precision cut and CNC-machined grafts the change in dimensions can cause problems with the grafts properly interfacing with the surgical instruments and fitting into the prepared surgical site.

To maintain full biomechanical strength and toughness of grafts, some have utilized an alternative preservation method of freezing cortical bone grafts, ligaments, tendons, and other soft tissue, such as costal cartilage, at −40° C. or less and then thawing the grafts in normal saline at the time of surgery. Thawing takes about 1 to 5 minutes with small grafts, such as those used for structural interbody support in the spine, or about 10 to 60 minutes for larger grafts. However, this preservation method requires that a validated and continuously monitored −80° C. freezer be present at any location where these grafts are to be stored. Alternatively, the grafts can be shipped on dry ice to the location where the grafts are to be used, and can then be returned to the tissue bank or other storage facility on dry ice if the grafts are not implanted. This method is therefore relatively complex and expensive, and is not feasible in all locations.

Hence, there remains a need for simple and cost effective systems and methods for storing and preserving implantable devices such as tissue grafts at an ambient temperature range. There also remains a need for systems and methods for storing and preserving implantable devices such as tissue grafts that do not affect the strength, shape, and dimensions of the grafts.

### SUMMARY OF THE INVENTION

The present invention includes systems for packing and storing implantable devices such as tissue for future use in a medical procedure. As discussed herein, embodiments of the present invention allow for tissue such as allograft to be effectively placed in a container and remain hydrated during storage. In embodiments where the tissue is an implant, the present invention allows for the tissue to simply be removed from the container and used in the medical procedure.

One aspect of the present invention is to provide a solution for storing an implantable device such as a tissue graft in one embodiment, the solution comprises water and an additional substance selected from calcium chloride, sodium bicarbonate, or a combination of both.

Another aspect of the present invention is to provide an implantable device such as a tissue graft for use as a surgical implant. In one embodiment, a bone allograft is provided, sealed in a container with a volume of a solution of the present invention, and maintained in a hydrated state until its use in surgery. In another embodiment, a bone allograft is provided, sealed in a container with a volume of a solution of the present invention and this container is further sealed in an outside container.

Another aspect, of the present invention is to provide a tissue graft system with a shelf life of up six months, a year, and even up to six years or more.

Another aspect of the present invention is to provide a system for storing an implantable device such as tissue prior to a surgical implant procedure, comprising a sealable container and housed therein: a volume of solution, the solution comprising water/saline, and calcium chloride or sodium bicarbonate; and said implantable device housed in the container and suspended in the solution.

Another aspect of the present invention is to provide a system for storing an implantable device such as tissue prior to a surgical implant procedure, comprising: a first sealable container, and housed therein a second sealable container; wherein the second sealable container houses a volume of solution and tissue at least partially submerged in the solution.

Another aspect of the present invention is to provide a method for providing an implantable device such as tissue for use in surgery, comprising: processing a sample of tissue to form a tissue implant that is dimensioned for implanting in a mammalian body; providing an inside sealable container; providing an outside sealable container; providing a tissue storage solution; sealing the tissue implant and the tissue storage solution in the inside container; and sealing the inside container inside the outside container.

Another aspect of the present invention is to provide a method of performing a medical device implant procedure, comprising: opening a first container, having a second container sealed therein; opening said second container having an implantable device such as a tissue implant stored therein, the tissue implant being hydrated in a solution and ready for implantation in a mammalian body; removing said tissue implant and implanting the tissue implant into said body.

### BRIEF DESCRIPTION OF THE FIGURES

FIG. 1 shows an example of a packaging system of the present invention. The outer container is resting in the inner container, and the top of the outer container is partially peeled away.

FIG. 2 shows an example of an inner container above an outer container.

FIG. 3 shows an example of the system of the present invention. It is a cutaway view showing the inner container resting in the outer container. It contains a solution of the present invention as well as a tissue implant of the present invention.

FIG. 4 is a graph that shows that no statistical differences were found in comparing the resulting mechanical properties of cortical bone preserved by the different preservation methods within each donor's data. The elastic modulus (megapascals/percent strain, MPa/%) for the two donors is shown in FIG. 4 with D1 being Donor 1 and D2 being Donor 2. The elastic modulus is the slope of the stress-strain curve.

FIG. 5 is a graph that shows the Yield Strength (megapascals, MPa) versus preservation method which is the first deflection from elastic deformation into the region of plastic deformation. No statistical differences were found in comparing the different preservation methods.

FIG. 6 is a graph that shows the ultimate compressive strength (maximum stress attained) (megapascals, MPa) versus preservation method for the two donors tested. This is the stress required to initiate fracture of the specimen. No statistical differences were found in comparing the different preservation methods.

FIG. 7 is a graph that shows the toughness versus preservation method for the six donors. Toughness is defined as the work (energy) required to fracture the graft (MJ/m3). No statistical differences were found in comparing the different preservation methods.

FIG. 8 is a graph that shows the strain (%) at the yield point versus preservation method. No statistical differences were found in comparing the different preservation methods.

FIG. 9 is a graph that shows the elastic modulus (megapascals/percent strain, MPa/%) versus preservation method for six donors, D3 through D8. No statistical differences were found between the isotonic sodium chloride (SC) groups and the isotonic calcium chloride (CC) groups.

FIG. 10 is a graph that shows the yield strength versus preservation method which is the first deflection from elastic deformation into the region of plastic deformation. No statistical differences were found between the isotonic sodium chloride (SC) groups and the isotonic calcium chloride (CC) groups.

FIG. 11 is a graph that shows the ultimate compressive strength (maximum stress attained) (megapascals, MPa) versus preservation method. This is the stress required to initiate fracture of the specimen. No statistical differences were found between the isotonic sodium chloride (SC) groups and the isotonic calcium chloride (CC) groups.

FIG. 12 is a graph that shows the toughness versus preservation method. Toughness is defined as the work (energy) required to fracture the graft (MJ/m3). No statistical differences were found between the isotonic sodium chloride (SC) groups and the isotonic calcium chloride (CC) groups.

FIG. 13 is a graph that r(megapascals, MPa) eviews calcium leaching study with isotonic sodium chloride preservation solution. The differences between the initial calcium levels and the calcium levels in the preservation solutions are highly statistically significant.

FIG. 14 is a graph that shows calcium leaching with isotonic sodium bicarbonate preservation solution. The differences between the initial calcium levels and the calcium levels in the preservation solutions are not statistically significant. Note that the initial calcium levels in the preservation solution from Donor 1 were below the quantifiable limit.

FIG. 15 is a graph that shows calcium leaching study with isotonic calcium chloride preservation solution. The differences between the initial calcium levels and the calcium levels in the preservation solutions after 90 days are not statistically significant.

FIG. 16-19 are graphs that show the bound water, pore water and the peak stress and toughness (from Experiment 1, the mechanical testing experiments discussed earlier).

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter describes solutions for storing tissue grafts (allografts, autografts, and xenografts), medical devices and biologics. In some embodiments the present solutions can store tissue grafts in a state suitable for implantation or substantially suitable for implantation. In some embodiments the solutions can store tissue grafts in a hydrated state, and in some instances can store tissue grafts, medical devices and biologics such that they do not need to be hydrated prior to implantation. In some embodiments the present solutions can store tissue grafts, medical devices and biologics such that their strength and ductility (i.e., toughness) is not compromised to the same extent as it is by certain known storage systems and methods. Furthermore, in some instances the present solutions can store tissue grafts, medical devices and biologics such that fluctuations in the shape and size of the grafts are minimized or eliminated. Further still, in some embodiments the present solutions can store tissue grafts, medical devices and biologics at temperatures above freezing, at ambient temperature, and/or at atmospheric pressure.

The term “implant,” or “implantable device” is to be broadly construed, particularly related to devices that are hydrated before implantation. Embodiments of the present invention include tissue, medical devices, biologics, etc.

Medical devices of the present invention include devices made of plastics, ceramics, metals, tissues (and combinations thereof) designed for the diagnosis or treatment of pathology or trauma. As indicated above, examples of medical devices can contain tissue, e.g. a bone/titanium hybrid. These hybrids are termed as Section 351 tissues (Public Health Service Act).

An example of biologics of the present invention include cultured autologous chondrocytes.

The form “tissue” is used herein to refer to a population of cells and the surrounding biological matrix (e.g. bone, cartilage, collagen in the skin, etc.). In some instances the tissue generally consists of cells of the same kind that perform the same or similar functions. The types of cells that make the tissue are not limited. In some embodiments tissue is part of a living organism, and, in some embodiments, tissue is excised from a living organism or artificial tissue. In some embodiments tissue can be part of bone, such as cortical bone, cancellous bone, or combinations thereof. In some instances the tissue can include soft tissue, such as costal cartilage, ligament tissue, tendon tissue, skin tissue, organ tissue, or the like.

In other instances, the tissue may be artificial. For example, the tissue can be an implantable product that promotes bone or other tissue growth. Thus, embodiments if the present invention include implantable collagen matrices. Other embodiments of the present invention include tissue growth promoting implants produced by 3D printing.

In other embodiments of the present invention, the system described herein can be used to package and store medical devices that require implantation.

As used herein, the terms “graft,” “tissue graft,” and even “tissue” are used interchangeably to generally refer to any tissue transplant or transfer. The term graft is inclusive of, but is not limited to, allografts, xenografts, autografts, and the like. Tissue transplanted from one person to another (i.e. within the same species) is termed as allograft; tissue transplanted from one species to an animal of another species is termed as xenografts, such as pig heart valves used to surgically replace a human's heart valve. Tissue transplanted from the patient to a surgical site in the same patient is termed as autograft such as skin grafting (from the patient) for the treatment of burns in that same patient. Those of ordinary skill will also appreciate that the term graft, as used herein, is inclusive of various different grafts, such as cortical and/or cancellous bone grafts, ligament tissue grafts, tendon tissue grafts, cortical cartilage tissue grafts, organ tissue grafts, skin tissue grafts, decellularized grafts and the like. In some instances the graft is implanted into a subject in need thereof to treat a particular condition. In some instances a graft will become calcified, ossified, incorporated, and/or vascularized after being implanted in a subject. The present grafts can include aseptically processed grafts, which may have been stored in refrigerated conditions.

Embodiments of the present invention include systems for packaging and housing the implantable device and solution described herein. These embodiments include glass containers, plastic containers, metal containers, metal foil containers, plastic-plastic pouch material (clear durable plastic) and combinations thereof (such as a glass container sealed with metal foil).

In some embodiments the systems comprise a plastic container that includes a plastic seal (i.e., plastic-plastic container) or a foil seal (i.e., plastic-foil container). Specific embodiments of containers comprise a polyethylene terephthalate glycol-modified polymer (PETG), a clear amorphous thermoplastic that can be injection molded or sheet extruded. In some embodiments the container includes a tray that is enclosed by a sealable lid. In some instances the lid can be peeled from the container.

Some embodiments of the present systems comprise a multiple-container system that comprise a plurality of containers. Exemplary multiple-container systems include two container systems. In some embodiments a two container system includes a first container, which houses the solution or houses the solution and the tissue graft, that is itself housed within a second container. The first container and the second container can be made from the same materials or can be made from different materials. Multiple-container systems can further ensure that the solution and the tissue graft are not exposed to a potentially contaminating environment. The second container housing the first container can protect the exterior of the first container from contacting potential contaminants. Thus, when opening the containers one can further protect the solution and the tissue graft housed in the containers from contaminants that potentially have contacted or become adhered to the exterior side of the first container.

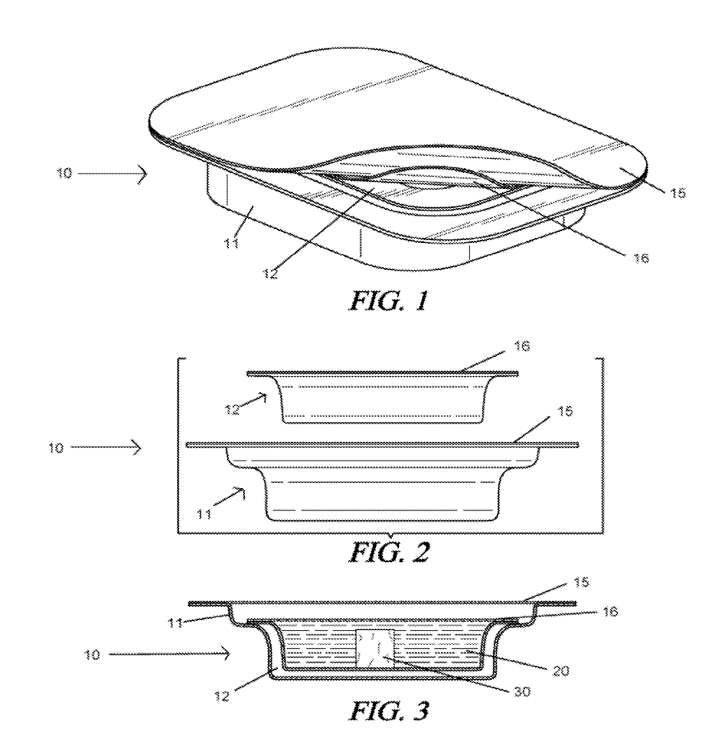

FIGS. 1-3 show an embodiment of the present invention in which the tissue is packaged and ready for use in surgery. The system 10 comprises a first, or outside container 11 and a second, inside container 12. The second container nests within the first. Each container has a removable air-tight seal 15, 16. For illustration purposes, the second contains holds the solution of the present invention 20, and submerged therein allograft30. The allograft is sealed, stored, and hydrated. The first and second containers of this embodiment allow for the second container to be removed from the first container and placed into the sterile field.

Due to this and other packaging methods of the present invention, the allograft or other applicable tissue is hydrated and ready to be surgically inserted into the recipient. The tissue may be stored at room temperature prior to the procedure, and does not require thawing or hydration.

In some embodiments the present solution for storing an implantable device of the present invention is an aqueous solution that comprises one or more additional substances. The present solutions can include water that is deionized water, distilled water, sterile water, or the like. In some instances the additional substances in the aqueous solution include a salt. In specific embodiments the solutions comprise sodium bicarbonate (NaHCO₃), calcium chloride (CaCl₂), or a combination thereof. In some embodiments the present solutions do not comprise saline. In some embodiments herein, reference to a substance, such as a salt, also includes ions of that salt formed in aqueous solutions. For instance, CaCl₂ can be inclusive of Ca²⁺ and Cl⁻ ions.

In this regard, the concentration of the additional substances in the solution can vary depending on the type of tissue graft, the size of the tissue graft, the intended purposes of the tissue graft, and/or the intended subject for the tissue graft. In some embodiments the solutions can comprise about 0.05 wt % to about 50 wt % of the additional substances. Thus, in certain embodiments the solutions can comprise about 0.05 wt %, 0.10wt %, 0.20 wt %, 0.30 wt %, 0.40 wt %, 0.50 wt %, 0.60 wt %, 0.70 wt %, 0.80 wt %, 0.90 wt %, 1.00 wt %, 1.50 wt %, 2.00 wt %, 2.50 wt %, 3.00 wt %, 3.50 wt %, 4.00 wt %, 4.50 wt %, 5.00 wt %, 6.00 wt %, 7.00 wt %, 8.00 wt %, 9.00 wt %, 10.00 wt %, 11.00 wt %, 12.00 wt %, 13.00 wt %, 14.00 wt %, 15.00 wt %, 16.00 wt %, 17.00 wt %, 18.00 wt %, 19.00 wt %, 20.00 wt %, 21.00 wt %, 22.00 wt %, 23.00 wt %, 24.00 wt %, 25.00 wt %, 26.00 wt %, 27.00 wt %, 28.00 wt %, 29.00 wt %, 30.00 wt %, 31.00 wt %, 32.00 wt %, 33.00 wt %, 34.00 wt %, 35.00 wt %, 36.00 wt %, 37.00 wt %, 38.00 wt %, 39.00 wt %, 40.00 wt %, 41,00 wt %, 42.00 wt %, 43.00 wt %, 44.00 wt %, 45.00 wt %, 46.00 wt %, 47.00 wt %, 48.00 wt %, 49.00 wt % or 50.00 wt %. of the one or more additional substances, wherein the additional substances may be selected from calcium chloride, sodium bicarbonate, or combinations thereof. In some embodiments the solution is saturated with the one or more additional substances.

Embodiments of the present invention include water and/or saline, and calcium chloride (CaCl₂) or sodium bicarbonate (NaHCO₃) concentrations. In embodiments, the solution comprises about 0.5-2.5 wt. % Calcium Chloride (CaCl₂). In other embodiments, the solution comprises about 0.5 to about 1.5 wt. % CaCl₂.

In other embodiments of the present invention, the solution comprises about 0.5-2.5 wt % of Sodium Bicarbonate (NaHCO₃). In other embodiments, the solution comprises about 0.5 to 1.5 wt % NaHCO₃.

Examples of ranges of solute concentration, including CaCl₂ and NaHCO₃ are from about 0.01% to 50% with 10% or less intervals. For example, the solute concentration may be about 0.01 wt %, 0.011 wt %, 0.012 wt %, 0.013 wt %, 0.014 wt %, 0.015 wt %, 0.016 wt %, 0.017 wt %, 0.018 wt %, 0.019 wt %, 0.02 wt %, 0.021 wt %, 0.022 wt %, 0.023 wt %, 0.024 wt %, 0.025 wt %, 0.026 wt %, 0.027 wt %, 0.028 wt %, 0.029 wt %, 0.03 wt %, 0.031 wt %, 0.032 wt %, 0.033 wt %, 0.034 wt %, 0.035 wt %, 0.036 wt %, 0.037 wt %, 0.038 wt %, 0.039 wt %, 0.04 wt %, 0.041 wt %, 0.042 wt %, 0.043 wt %, 0.044 wt %, 0.045 wt %, 0.046 wt %, 0.047 wt %, 0.048 wt %, 0.049 wt %, 0.05 wt %, 0.051 wt %, 0.052 wt %, 0.053 wt %, 0.054 wt %, 0.055 wt %, 0.056 wt %, 0.057 wt %, 0.058 wt %, 0.059 wt %, 0.06 wt %, 0.061 wt %, 0.062 wt %, 0.063 wt %, 0.064 wt %, 0.065 wt %, 0.066 wt %, 0.067 wt %, 0.068 wt %, 0.069 wt %, 0.07 wt %, 0.071 wt %, 0.072 wt %, 0.073 wt %, 0.074 wt %, 0.075 wt %, 0.076 wt %, 0.077 wt %, 0.078 wt %, 0.079 wt %, 0.08 wt %, 0.081 wt %, 0.082 wt %, 0.083 wt %, 0.084 wt %, 0.085 wt %, 0.086 wt %, 0.087 wt %, 0.088 wt %, 0.089 wt %, 0.09 wt %, 0.091 wt %, 0.092 wt %, 0.093 wt %, 0.094 wt %, 0.095 wt %, 0.096 wt %, 0.097 wt %, 0.098 wt %, 0.099 wt %, 0.1 wt %, 0.11 wt %, 0.12 wt %, 0.13 wt %, 0.14 wt %, 0.15 wt %, 0.16 wt %, 0.17 wt %, 0.18 wt %, 0.19 wt %, 0.2 wt %, 0.22 wt %, 0.24 wt %, 0.26 wt %, 0.28 wt %, 0.3 wt %, 0.32 wt %, 0.34 wt %, 0.36 wt %, 0.38 wt %, 0.4 wt %, 0.42 wt %, 0.44 wt %, 0.46 wt %, 0.48 wt %, 0.5 wt %, 0.55 wt %, 0.6 wt %, 0.65 wt %, 0.7 wt %, 0.75 wt %, 0.8 wt %, 0.85 wt %, 0.9 wt %, 0.95 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4 wt %, 4.1 wt %, 4.2 wt %, 4.3 wt %,4.4 wt %, 4.5 wt %, 4.6 wt %, 4.7 wt %, 4.8 wt %, 4.9 wt %, 5 wt %, 5.25 wt %, 5.5 wt %, 5.75 wt %, 6 wt %, 6.25 wt %, 6.5 wt %, 6.75 wt %, 7 wt %, 7.25 wt %, 7.5 wt %, 7.75 wt %, 8 wt %, 8.25 wt %, 8.5 wt %, 8.75 wt %, 9 wt %, 9.25 wt %, 9.5 wt %, 9.75 wt %, 10 wt %, 10.25 wt %, 10.5 wt %, 10.75 wt %, 11 wt %, 11.25wt %, 11.5wt %, 11.75 wt %, 12 wt %, 12.25 wt %, 12.5 wt %, 12.75 wt %, 13 wt %, 13.25 wt %, 13.5 wt %, 13.75 wt %, 14 wt %, 14.25 wt %, 14.5 wt %, 14.75 wt %, 15 wt %, 15.25 wt %, 15.5 wt %, 15.75 wt %, 16 wt %, 16.25 wt %, 16.5 wt %, 16.75 wt %, 17 wt %, 17.25 wt %, 17.5 wt %, 17.75 wt %, 18wt %, 18.25 wt %, 18.5 wt %, 18.75 wt %, 19 wt %, 19.25 wt %, 19.5 wt %, 19.75 wt %, 20 wt %, 20.25 wt %, 20.5 wt %, 20.75 wt %, 21 wt %, 21.25 wt %, 21.5 wt %, 21.75 wt %, 22 wt %, 22.25 wt %, 22.5 wt %, 22.75 wt %, 23 wt %, 23.25 wt %, 23.5 wt %, 23.75 wt %, 24 wt %, 24.25 wt %, 24.5 wt %, 24.75 wt %, 25 wt %, 25.25 wt %, 25.5 wt %, 25.75 wt %, 26 wt %, 26.25 wt %, 26.5 wt %, 26.75 wt %, 27 wt %. 27.25 wt %, 27.5 wt %, 27.75 wt %. 28 wt %, 28.25 wt %, 28.5 wt %, 28.75 wt %, 29 wt %, 29.25 wt %, 29.5 wt %, 29.75 wt %, 30 wt %, 30.25 wt %, 30.5 wt %, 30.75 wt %, 31 wt %, 31.25 wt %, 31.5 wt %, 31.75 wt %, 32 wt %, 32.25 wt %, 32.5 wt %, 32.75 wt %, 33.25 wt %, 33.5 wt %, 33.75 wt %, 34 wt %, 34.25 wt %, 34.5 wt %, 34.75 wt %, 35 wt %, 35.25 wt %, 35.5 wt %, 35.75 wt %, 36 wt %, 36.25 wt %, 36.5 wt %, 36.75 wt %, 37 wt %, 37.25 wt %, 37.5 wt %, 37.75 wt %, 38 wt %, 38.25 wt %, 38.5 wt %, 38.75 wt %, 39 wt %, 39.25 wt %, 39.5 wt %, 39.75 wt %, 40 wt %, 40.25 wt %, 40.5 wt %, 40.75 wt %, 41 wt %, 41.25 wt %, 41.5 wt %, 41.75 wt %, 42 wt %, 42.25 wt %, 42.5 wt %, 42.75 wt %, 43 wt %, 43.25 wt %. 43.5 wt %, 43.75 wt %, 44 wt %, 44.25 wt %, 44.5 wt %, 44.75 wt %, 45 wt %, 45.25 wt %, 45.5 wt %, 45.75 wt %, 46 wt %, 46.25 wt %, 46.5 wt %, 46.75 wt %, 47 wt %, 47.25 wt %, 47.5 wt %, 47.75 wt %, 48 wt %, 48.25 wt %, 48.5 wt %, 48.75 wt %, 49 wt %, 49.25 wt %, 49.5 wt %, 49.75 wt % or 50 wt %.

In other embodiments the solutions are “isotonic,” which, as used herein, refers to solutions that exert zero osmotic pressure on the tissue, biologic or device. In some instances isotonic solutions provide a balance between under-hydrating and over-hydrating a tissue graft. Furthermore, in some instances the net water exchange between a tissue graft and an isotonic solution will be substantially zero. Accordingly, relatively less concentrated (i.e., hypotonic) solutions over-hydrate a tissue graft, whereas relatively more concentrated (i.e., hypertonic) solutions can cause under-hydration of a tissue graft,

As described herein, in some embodiments the implantable device (such as, for example, a tissue graft), solution, and/or system are sterile. In some embodiments the tissue graft, solution, and/or system are sterilized before and/or after packaging the tissue graft and solution in a system. Two methods exemplary sterilization methods include gamma irradiation and electron beam (e-beam) sterilization. Electron beam is a high flux highly charged electron stream generated by an accelerator that may be either pulsed or continuous. The electrons destroy chemical bonds and thus can destroy DNA and RNA, which can prevents microorganisms from reproducing. High-energy electron beams can penetrate a system and a tissue graft. In certain embodiments e-beam is selected as a sterilization method for systems and tissue grafts that have relatively low, uniform densities. In some instances e-beam is cost-effective but potentially more sensitive to the product/package combination as compared to gamma irradiation. In this respect, gamma irradiation penetrates dense materials which allows for greater variance in density (e.g., systems and tissue grafts with non-uniform density). Gamma irradiation can come from radioactive sources, such as Cobalt-60 or Cesium-137. Gamma irradiation can have a high tolerance to inhomogeneity and also have high penetration into the product.

The presently-disclosed subject matter further includes methods for using the present solutions and systems for storing implantable devices such as tissue grafts. In some embodiments the tissue grafts can be stored in the present solutions and systems for up to about 6 months or longer, including up to a year or even up to about 6 years. In some embodiments the tissue grafts can be stored for more than 5 years. In some embodiments the temperature at which the tissue grafts must be stored is not particularly limited. In specific embodiments the tissue grafts can be stored at about 2° C. to about 40° C.

### EXAMPLES

The presently-disclosed subject matter is further illustrated by the following specific but non-limiting examples.

While the terms used herein axe believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a container” includes a plurality of such containers, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter. For example, if the value “10” is disclosed, then “about 10” is also disclosed. Ranges can also be expressed as from “about” one particular value, and/or to “about” another particular value. It is also understood that each unit between two particular units are also disclosed. For example, if 10 and 15 are disclosed, then 11, 12, 13, and 14 are also disclosed.

As used herein, the term “about,” when referring to a value or to art amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

This example includes embodiments of the present invention as well as controls and comparitors. Particularly, there are seven solutions total, 3 concentrations each of calcium chloride and sodium bicarbonate, a control, 0.9% normal saline and a comparator which was lyophilized (freeze dried) preservation. The lyophilized samples represent the standard preservation method across the tissue industry. CNC machined cylinders of cortical bone (6 mm in diameter and 2 mm in diameter), were placed in sealable containers (one of each) for the aging study. Each container contained one mechanical testing cylinder, 6 mm diameter, and at least one NMR cylinder, 2 mm in diameter. The eight preservation methods are listed below.

1. 0.57% Calcium chloride

2. 1.14% Calcium chloride (isotonic)

3. 2.28% Calcium chloride

4. 0.65% Sodium bicarbonate

5. 1.29 % Sodium bicarbonate (isotonic)

6. 2.60% Sodium bicarbonate

7. CONTROL: 0.9% Sodium Chloride (isotonic)

8. COMPARITOR: Lyophilized (freeze-dried) samples.

All donors utilized have provided research consent. All specimens were fully processed including all washing, disinfecting steps and gamma irradiation sterilization as if they were transplantable allografts.

Mechanical Testing Summary

Human allograft tissue specimens were prepared from eight human tissue allograft donors.

Cylindrical test specimens were CNC machined with machined tops and bottoms to be perpendicular to longitudinal axis of specimen. Dimensions were 6 mm O (diameter)/approximately 6 mm height. The machining was performed to a high degree of precision. Tolerances on the diameter were within 0.01 mm and the flatness of the tops and bottom surfaces of the test allografts were to within 0.02 mm.

Specimen height was measured before each individual mechanical test and utilized as the gauge length of that particular test.

Load to Failure:

As per ASTM 2077, the allograft was inserted between two stainless steel blocks with parallel surfaces,

Teflon tape was placed on the top and bottom of the allograft cylinder to minimize friction between the platen and bone.

The allograft was pre-loaded to 10 N compression to allow for solid placement between the platens and,

Then loaded to failure in position control at 12.7 mm/rain or until the load drops so as to signify failure. ASTM 2077 states, “at a rate no greater than 25 mm/min, upon which the graft shows signs of failure (cracking or collapse).”

Data Analysis:

The load versus displacement data was used to calculate true stress/true strain using the platen distance for displacement, 28.27 mm² as the cross-sectional area and the gauge length as measured for each individual specimen.

In doing this the assumption was made that the bone does not barrel but will maintain volume constancy and the expansion was uniform (until ultimate compressive strength is attained). True stress/true strain curves were generated for each test and compressive modulus, yield strength and ultimate compressive strength were determined if possible. As can be the case with compression tests, there may be no distinct failure point.

It should be noted that the failure stress of the lumbar vertebral endplate (without aggressive endplate removal—just cutting back to the bony endplate) is approximately 17.4-20.0 MPa (Lowe, et. al. Spine Vol. 29, No 21, pp. 2389-2394. 2004). The cortical bone (of the allograft) should be stronger than the endplate. Note that the endplate anatomically are dense cancellous bone as opposed to normal cancellous bone of moderate density.

Results:

The first study involved the preparation of two donors each with 40 specimens, 5 each representing the eight aforementioned groups of different preservation methods, aqueous solutions or concentrations with the control groups being the 0.9% normal saline groups and the lyophilized (freeze-dried) groups. The allograft specimens were not aged and were tested within weeks after manufacturing, processing, packaging and gamma irradiation.

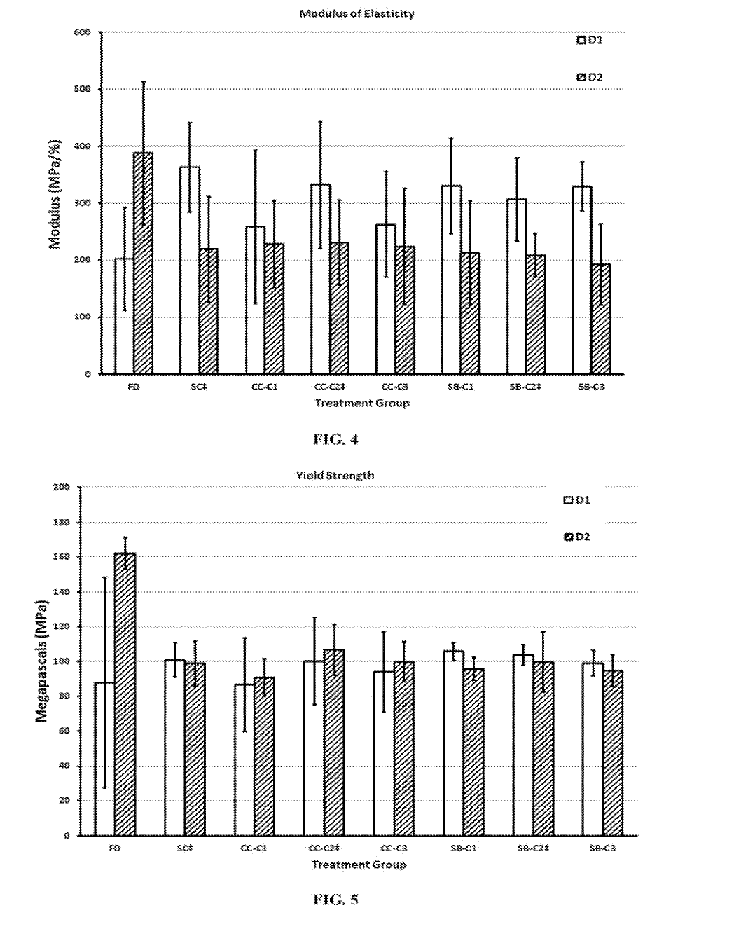

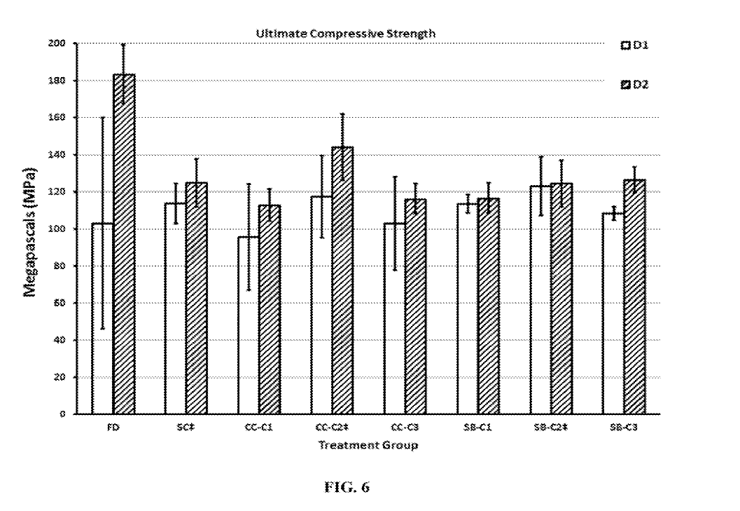

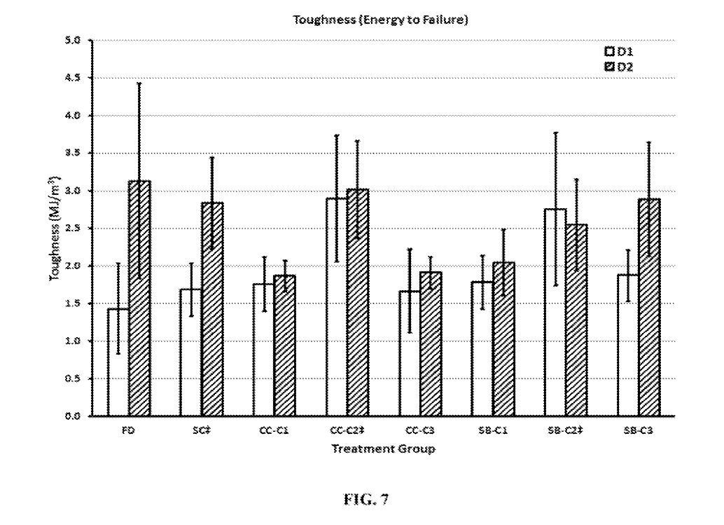

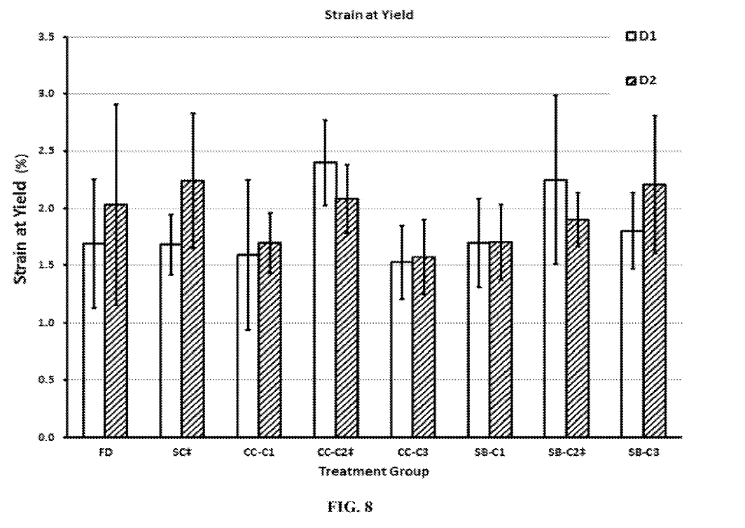

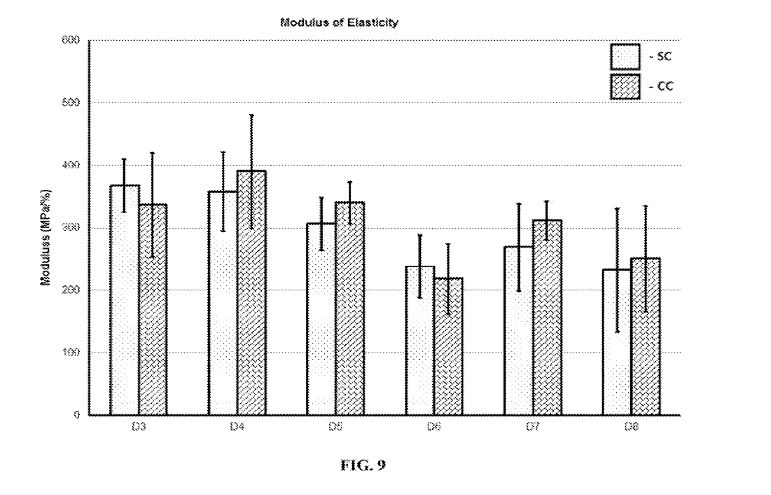

No statistical differences were found in comparing the different preservation methods within each donor's data. The elastic modulus for the two donors is shown in FIG. 4 with D1 being Donor 1 and D2 being Donor 2. The elastic modulus is the slope of the stress-strain curve. FIG. 5 shows the Yield Strength versus preservation method for the six donors which is the first deflection from elastic deformation into the region of plastic deformation. FIG. 6 shows the ultimate compressive strength (maximum stress attained) for the six donors versus preservation method. This is the stress required to initiate fracture of the specimen. FIG. 7 shows the toughness versus preservation method. Toughness is defined as the work (energy) required to fracture the graft. FIG. 8 shows the strain at the yield point versus preservation method. The legend for the preservation groups in the graphs are as follows:

FD: Lyophilized (freeze-dried) samples, control group.

SC: 0.9% Sodium Chloride (isotonic), control group.

CC-C1: 0.57% Calcium chloride

CC-C2: 1.14% Calcium chloride (isotonic)

CC-C3: 2.28% Calcium chloride

SB-C1: 0.65 % Sodium bicarbonate

SB-C2: 1.29 % Sodium bicarbonate (isotonic)

SB-C3: 2.60% Sodium bicarbonate

The second experiment, a comparison on isotonic (0.9%) aqueous sodium chloride with aqueous isotonic calcium chloride (1.14%), involved the preparation, of six donors each with 24 specimens each, 12 in isotonic sodium chloride and 12 in isotonic calcium chloride. Each bar in each graph represents the 12 allografts in that group.

The allograft specimens were not aged and were tested within weeks after manufacturing, processing, packaging and gamma irradiation. Again, no statistical differences in intrinsic mechanical properties were found between the donors and in comparing the two different preservation methods.

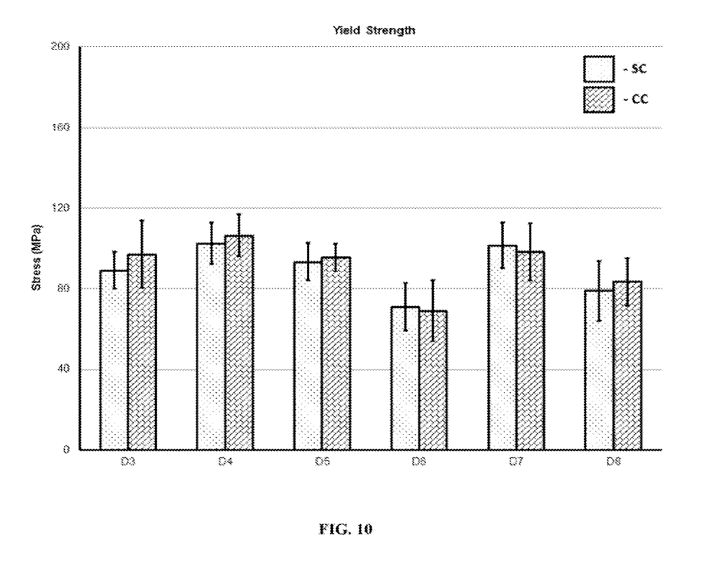

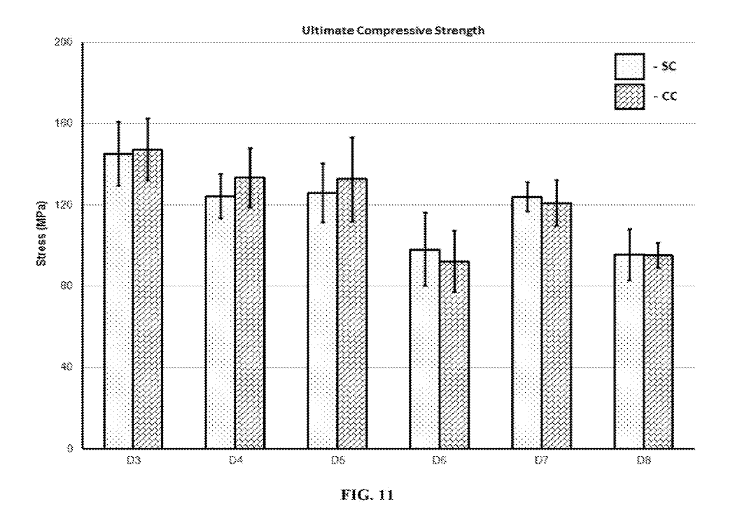

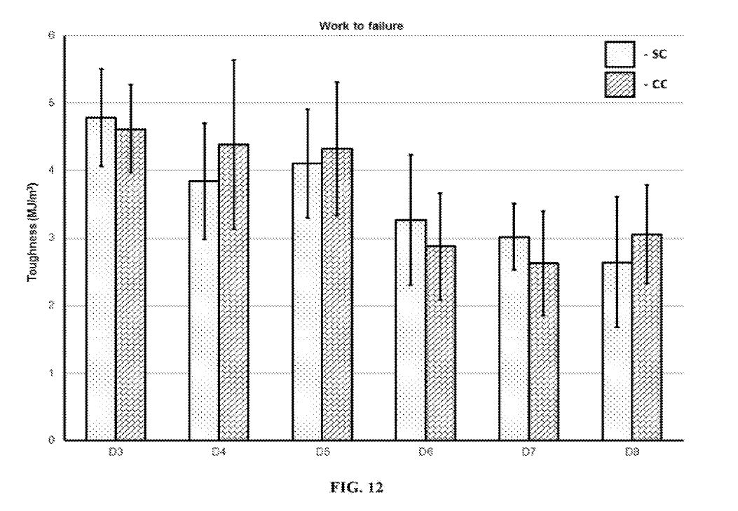

The elastic modulus for the six donors , D3 to D8 is shown in FIG. 9. The elastic modulus is the slope of the stress-strain curve. FIG. 10 shows the Yield Strength versus preservation method which is the first deflection from elastic deformation into the region of plastic deformation. FIG. 11 shows the ultimate compressive strength, (maximum stress attained) versus preservation method. This is the stress required to initiate fracture of the specimen. FIG. 12 shows the toughness versus preservation method. Toughness is defined as the work (energy) required to fracture the graft.

Each donor had 24 specimens machined to cylindrical dimensions (12 were preserved in isotonic sodium chloride and 12 in isotonic calcium chloride). No statistical differences between the different preservation methods in terms of intrinsic mechanical properties were shown.

Calcium Leaching Analysis Summary

One significant consequence of preserving cortical bone or other tissue within an aqueous media is the loss of calcium (and other components) by leaching. This can occur by demineralization and by other processes and is driven by thermodynamics (concentration gradients).

Thirty allografts each from two donors were prepared as per the aforementioned procedure (6 mm diameter cortical dowels) to compare the calcium leaching effects of isotonic calcium chloride, isotonic sodium bicarbonate with isotonic sodium chloride being the control group. The aqueous preservation solutions had the calcium analysis assessed by inductively coupled plasma—optical emission spectroscopy (ICP-OES). This test for calcium and other ions and metals is far more sensitive than atomic absorption spectroscopy (AAS).

The graft specimens were manufactured, processed, packaged and then sterilized with gamma irradiation. Half of the allografts from each donor (5 from each group) had calcium analysis performed on the storage solution right after terminal sterilization and the other 5 allografts were aged at room temperature (20° C., 68° F.) for 90 days. Then the calcium analysis on the preservation solution was performed. Negative controls (preservation solutions within the packaging that had no allograft included) were also tested.

Isotonic Sodium Chloride:

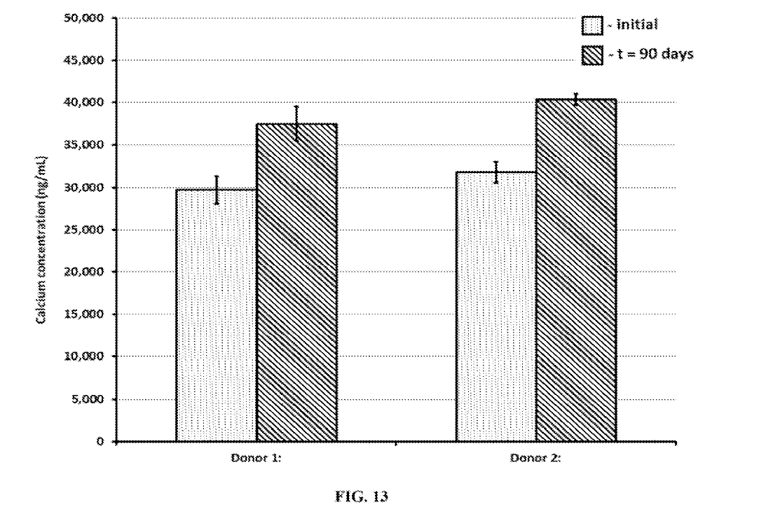

In the allografts that were preserved in isotonic sodium chloride, the solution in Donor 1, exhibited a 26.3% increase in the calcium in solution over 90 days which was highly statistically significant (p=0.006). The solution from the grafts of Donor 2, exhibited a 27.1% increase in the calcium in solution over 90 days which was highly statistically significant (p=0.00003). This means that some calcium leaching had occurred over the 90 day period. These data are shown in FIG. 13.

Isotonic Sodium Bicarbonate:

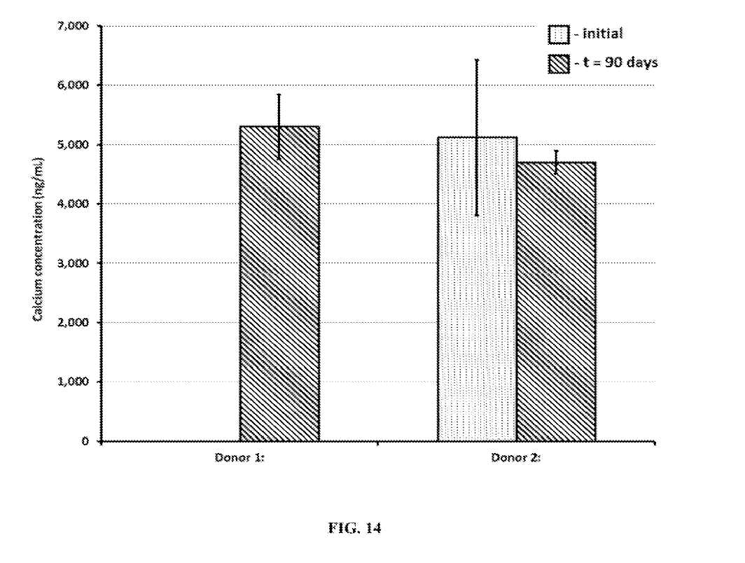

In the allografts that were preserved in isotonic sodium bicarbonate, the solution in Donor 1, had the initial calcium levels below the quantifiable limit (BQL) by the assay. The allografts that were aged for 90 days had an increase up to 5300 ng/mL. The solution from the grafts of Donor 2, showed there was an 8% decrease in the calcium in solution over 90 days which was not statistically significant (p=0.62). This means that in Donor 1 some calcium leaching had occurred over the 90 day period but that in Donor 2, no net calcium leaching occurred. These data are shown in FIG. 14.

Isotonic Calcium Chloride:

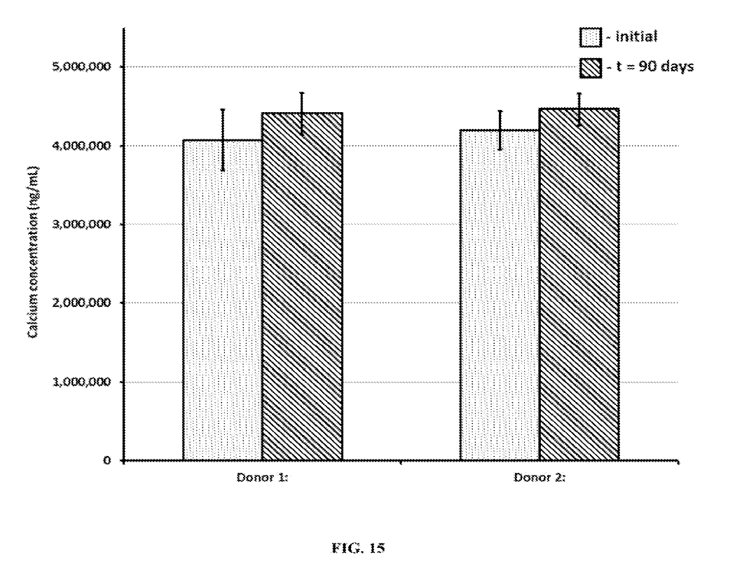

In the allografts that were preserved in isotonic calcium chloride, the solution in Donor 1, exhibited an 8.3% increase in the calcium in solution over 90 days which was not statistically significant (p=0.23). The solution from the grafts of Donor 2, exhibited a 6.4% increase in the calcium in solution over 90 days which again was not statistically significant (p=0.20). This means that no net calcium leaching occurred over the 90 day period. These data are shown in FIG. 15.

The isotonic calcium chloride preservation solution showed no net calcium leaching from the cortical bone allograft into the solution over the 90 day period while the isotonic sodium chloride solution with cortical bone allografts did show statistically significant increases in total calcium content (in the solution) over the 90 day period. The fact that the isotonic sodium chloride preservation solution had a highly statistically significant increase in calcium levels after only 90 clays of room temperature aging is an important finding because the shelf life of this tissue or other materials can be as long as four to six years or even longer.

Water Content of the Cortical Bone as a Function of Preservation Solution;

With the same grafts (additional CNC machined cylinders, 2 mm diameter) the mobile and bound water was assayed utilizing nuclear magnetic resonance. The proper hydration of biological tissues is a critical parameter in controlling the tissue's mechanical properties. Pathologic tissue such as that in plantar fasciitis and degenerated intervertebral discs typically exhibits less than optimal hydration.

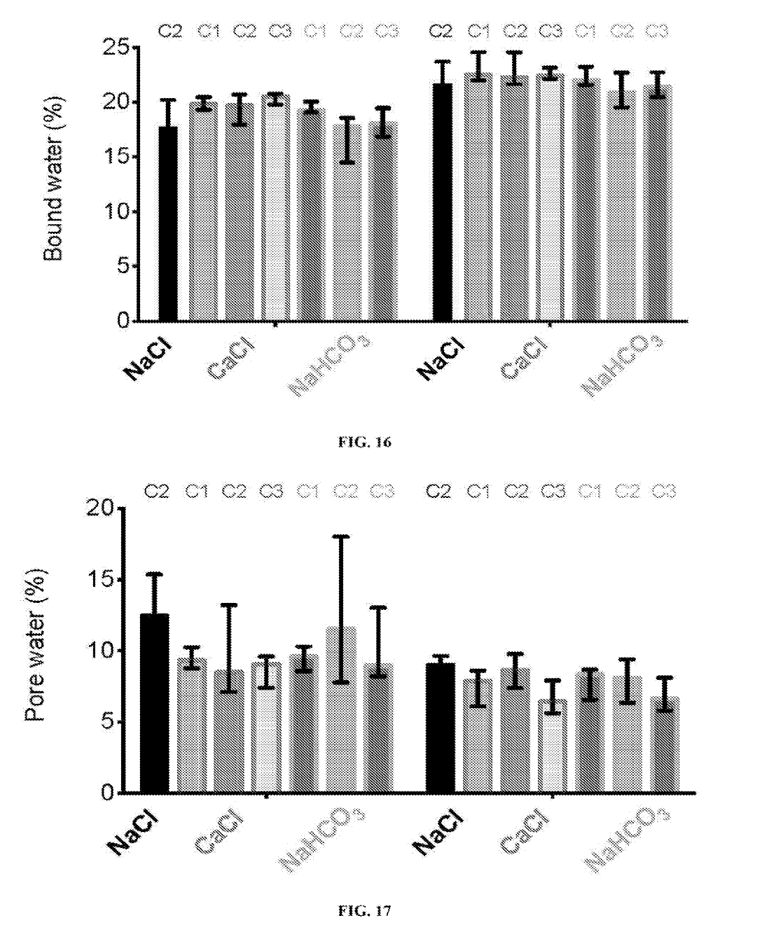

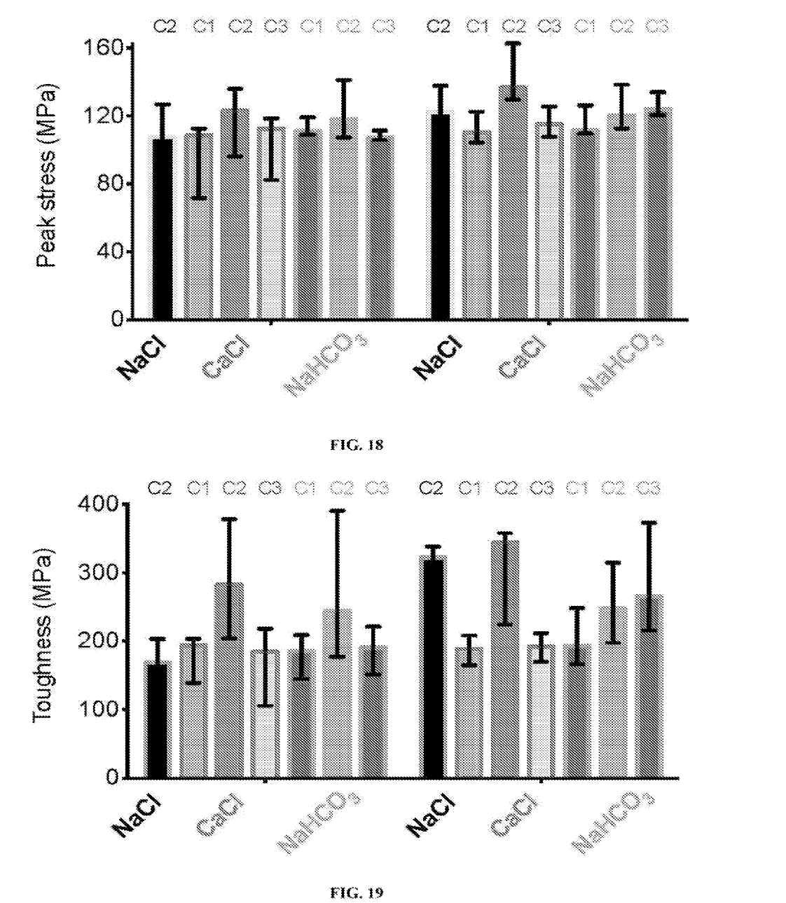

FIGS. 16, 17, 18 and 19. show the bound water, pore water and the peak stress and toughness (from Experiment 1, the mechanical testing experiments discussed earlier).

No statistically significant differences were found in graft hydration in both bound water and pore water in the solutions.

The invention thus being described in terms of a best mode for achieving said invention's objectives, it will be appreciated by one of ordinary skill in the art that variations of the invention may be made without deviating from the spirit and scope of the present invention.

We claim:

1. A system for storing an implantable device prior to a surgical implant procedure, comprising
a sealable container and housed therein:
a volume of solution, the solution comprising water, and calcium chloride or sodium bicarbonate; and
said implantable device housed in the container and suspended in the solution.

2. The system of claim 1, wherein the implantable device is a tissue graft.

3. The system of claim 1, wherein the implantable device is a tissue allograft.

4. The system of claim 1, wherein the solution comprises water and 0.5-10 wt. % calcium chloride.

5. A system for storing an implantable device prior to a surgical implant procedure, comprising:
a first sealable container, and housed therein a second sealable container; wherein
the second sealable container houses a volume of solution and said implantable device at least partially submerged in the solution.

6. The system of claim 5, wherein the solution comprises calcium chloride.

7. The system of claim 6, wherein the calcium chloride is present in an amount of from about 0.5-2.5 wt. %.

8. The system of claim 6, wherein the solution is isotonic.

9. The system of claim 5, wherein the solution comprises sodium bicarbonate.

10. The system of claim 9, wherein the sodium bicarbonate is present in an amount of from about 0.5-2.5 wt. %.

11. The system of claim 9, wherein the solution is isotonic.

12. The system of claim 5, wherein the implantable device is allograft.

13. The system of claim 12, wherein the allograft is a bone allograft for spinal fusion surgery, or other surgical procedure including for dental, dermatologic or for diagnostic purposes.

14. The system of claim 5, wherein the implantable device is a tissue-containing medical device.

15. The system of claim 5, wherein the implantable device is a cellular-based implantable device.

16. A method for providing an implant for use in surgery, comprising:
providing an implant that is dimensioned for implanting in a mammalian body;
providing an inside sealable container;
providing an outside sealable container;
providing an implant storage solution;
sealing the tissue implant and the tissue storage solution in the inside container;
sealing the inside container inside the outside container.

17. The method of claim 16, wherein the providing step comprises processing a sample of tissue to form a tissue implant that is dimensioned for implanting in a mammalian body.

18. The method of claim 17, wherein the tissue implant is a hone tissue graft.

19. The method of claim 17, wherein the bone tissue graft comprises cancellous hone, cortical bone, or a combination thereof.

20. The method of claim 16, wherein the implant is a medical device.

21. The method of claim 20, wherein the implant comprises a biological.

22. The method of claim 17, wherein the tissue implant includes costal cartilage, ligament tissue, tendon tissue, skin tissue, organ tissue, or a combination thereof.

24. The method of claim 16, wherein the solution comprises water and 0.5-20 wt. % calcium chloride.

25. The method of claim 16, wherein the solution comprises water and 0.5-10 wt. % calcium chloride.

26. The method of claim 16, wherein the solution comprises water and 0.5-5 wt. % calcium chloride.

27. The method of claim 16, wherein the solution comprises water and sodium bicarbonate.

28. The method of claim 16, wherein the solution comprises water and 0.5-20 wt. % sodium bicarbonate.

29. The method of claim 16, wherein the solution is isotonic.

30. A method of performing an implant procedure, comprising:
opening a first container, having a second container sealed therein;
opening said second container having an implant stored therein, the implant being hydrated in a solution and ready for implantation in a mammalian body;
removing said implant; and
implanting the implant into said body.

31. The method of claim 30, wherein the implant is a tissue implant.

32. The method of claim 31, wherein the tissue implant is a bone tissue graft.

33. The method of claim 32, wherein the bone tissue graft comprises cancellous bone, cortical bone, or a combination thereof.
